(19) United States
(12) Patent Application Publication
(10) Pub. No.: US 2009/0197955 A1
(43) Pub. Date: Aug. 6, 2009

(54) METHODS OF IMPROVING DHA DEPOSITION AND RELATED FUNCTION AND/OR DEVELOPMENT

(75) Inventors: Shawna L. Lemke, Webster Groves, MO (US); Gary F. Hartnell, St. Peters, MO (US)

Correspondence Address: Armstrong Teasdale LLP (29135);Christopher M. Goff, One Metropolitan Square, Suite 2600, St. Louis, MO, 63102-2740, US

(73) Assignee: Monsanto Company, St. Louis, MO (US)

(21) Appl. No.: 12/362,133

(22) Filed: Jan. 29, 2009

Related U.S. Application Data
(60) Provisional application No. 61/063,275, filed on Jan. 31, 2008.

(51) Int. Cl. A61K 31/202

(52) U.S. Cl. 514/560

(57) ABSTRACT

The present invention provides for improved deposition of DHA in human or animal tissues by incorporating healthy lipids containing stearidonic acid into humans or animal through prolonged food or feed, as well as through supplements, and therapeutics. Improved deposition of DHA through consumptions of edible compositions comprising stearidonic acid can be used to improve numerous indicators of health and development, including indicators of heart health, neurological function and development, and the Omega-3 Index in tissues.

### CROSS REFERENCE TO RELATED APPLICATIONS

This patent application claims priority from U.S. Provisional Patent Application 61/063,275 filed on Jan. 31, 2008, the entire contents of which is incorporated herein by reference.

### FIELD OF THE INVENTION

The invention relates to the enhancement of desirable characteristics in humans or animals and/or human or animal tissues through the incorporation of beneficial fatty acids in human food or animal feed or in human food or animal feed supplements. More specifically, the invention relates to deposition of DHA in various tissues and associated physiological improvements over time and multiple doses through applications of food, feed, supplements, and therapeutics comprising stearidonic acid.

### BACKGROUND OF THE INVENTION

The present invention is directed to a method for improving human or animal health through the utilization of plant-derived stearidonic acid (“SDA”) or SDA oil as a component in feed, food, supplements, and/or therapeutic compositions. Specifically, the inventors provide techniques and methods for the utilization of transgenic plant-derived SDA compositions in products that improve the physiological, intellectual, or emotional state of a human or animal through conversion of the SDA to docosahexaenoic acid (DHA) in vivo.

Many studies have made a physiological link between dietary fats and pathologies such obesity and atherosclerosis in humans. In some instances, human consumption of dietary fats has been discouraged by the medical establishment. More recently, the qualitative differences existing between dietary fats and their corresponding health benefits for consumers have begun to be recognized by physicians and nutritionists.

Recent studies have determined that despite their relatively simple biological structures there are some types of fats that, when consumed by humans or animals, improve physiological function in some ways and that may, in fact, be essential to certain physiological processes. The wider class of fat molecules includes fatty acids, isoprenols, steroids, other lipids and oil-soluble vitamins. Among these are the fatty acids. The fatty acids are carboxylic acids, which have from 2 to 26 carbon atoms in their molecular “backbone,” with few desaturated sites in their carbohydrate structure, many being fully hydrogenated. They generally have dissociation constants (pKa's) of about 4.5 indicating that in normal human physiological conditions (Es: normal human physiological pH is about 7.4) the vast majority will be in a dissociated form.

With continued experimentation researchers have begun to understand the nutritional need for fats and in particular fatty acids in the human diet and relative to supplementation in animal feed. For this reason, many in the food industry have begun to focus on providing the optimal fatty acid and lipid profiles in the production of food or feed, with its consequent benefits for the animals consuming the modified feed and especially in those products derived from those animals for human consumption. This focus has been particularly intense for the production and incorporation of Omega-3 fatty acids into the diet.

Omega-3 fatty acids are long-chain polyunsaturated fatty acids (18-22 carbon atoms in chain length) with the first of the double bonds (“unsaturations”) beginning with the third carbon atom from the methyl end of the molecule. They are called “polyunsaturated” because their molecules have two or more double bonds “unsaturations” in their carbohydrate chain. They are termed “long-chain” fatty acids since their carbon backbone has at least 18 carbon atoms. In addition to stearidonic acid “SDA” the omega-3 family of fatty acids includes alpha-linolenic acid (“ALA”), eicosatetraenoic acid (ETA), eicosapentaenoic acid (“EPA”), docosapentaenoic acid (DPA), and docosahexaenoic acid (“DHA”). ALA can be considered a “base” omega-3 fatty acid, from which EPA and DHA are made in the body through a series of enzymatic reactions, including the production of SDA. Most nutritionists point to DHA and EPA as the most physiologically important of the Omega-3 fatty acids with the most beneficial effects. However, SDA has also been shown to have significant health benefits. See for example, U.S. Pat. No. 7,163,960 herein incorporated by reference.

The biosynthetic pathway from ALA to longer chain fatty acids is called “elongation” (the molecule becomes longer by incorporating new carbon atoms) and “desaturation” (new double bonds are created), respectively. In nature, ALA is primarily found in certain plant leaves and seeds (e.g., flax) while EPA and DHA mostly occur in the tissues of cold-water predatory fish (e.g., tuna, trout, sardines and salmon), originating from the marine algae or microbes that they feed upon.

Along with the movement of food companies to develop and deliver essential fats and oils as an important components in a healthy human diet, governments have begun developing regulations pushing for the adoption of PUFA's in the diet. The difficulty in supplying these needs has been the inability to develop a large enough supply of Omega-3 oil to meet growing marketplace demand. As already mentioned, the Omega-3 fatty acids commercially deemed to be of highest value, EPA and DHA, also chemically oxidize very quickly over time limiting commercial availability and durability. Importantly, during the rapid process of EPA and DHA degradation these long chain fatty acids develop rancid and profoundly unsatisfactory sensory properties that make their inclusion in many foodstuffs difficult or impossible from a commercial acceptance perspective. In addition, with increased demand for Omega-3 fatty acids has come the realization that already depleted global fish stocks cannot meet any significant growth in future human nutritional needs for Omega-3's. These limitations on supply, durability, stability and sourcing greatly increase cost and correspondingly limit the availability of dietary Omega-3's.

Accordingly, a need exists to enhance and optimize the productivity of livestock animals. The compositions of the current invention comprise SDA compositions that can be used in producing food, feed, supplements and therapeutics.

In addition, a need exists to provide a consumer-acceptable means of delivering EPA and DHA or critical precursors in food formulations in a commercially acceptable way. The current invention provides an alternative to fish or microbe supplied Omega-3 fatty acids in the form of edible compositions comprising beneficial acids and does so utilizing a comparatively chemically stable fatty acid, SDA, as a source that offers improved cost-effective production and abundant supply as derived from transgenic plants.

According to preferred embodiments of the current invention, the preferred plant species that can be modified to supply demand are: soybeans, corn, and canola, but many other plants could also be included as needed and as scientifically practicable. Once produced the SDA of the invention can be used to improve the health characteristics of a great variety of food products. This production can also be scaled-up as needed to both reduce the need to harvest wild fish stocks and to provide essential fatty acid (FA) components for aquaculture operations, each greatly easing pressure on global fisheries and wild fish stocks.

Omega-3 fatty acids have been investigated as a potential way to improve performance and meat quality in livestock animals. Previous attempts to increase the concentration of beneficial fatty acids have included supplementing the diet with omega-3 fatty acids These methods include addition of highly unstable EPA or DHA which are less stable and more difficult to obtain; or incorporation of traditional omega-3 fatty acids such as alpha-linolenic acid (ALA), which are not converted to the beneficial forms efficiently enough to be practical. Nutritional studies have shown that, compared to ALA, SDA is 3 to 4 times more efficiently converted in vivo to EPA in humans (Ursin, 2003).

While conversion of SDA to EPA has been shown in numerous studies, conversion of SDA to DHA has not been shown. Rather, numerous studies have failed to find evidence of increased DHA concentrations in blood or tissues after feeding SDA. See for example, Harris et al. (2007) and James et al., (2003), herein incorporated by reference.

Surprisingly, the inventors have found that feeding humans or animals SDA compositions is highly effective in increasing the omega-3 fatty acid levels of SDA (18:4), ETA (omega-3 20:4), EPA (eicosapentaenoic acid), DPA (docosapentaenoic acid), DHA (docosahexaenoic acid) in some animal tissues including heart and brain tissues.

Conversion of SDA to DHA resulting in increased concentration of DHA in brain phospholipids is a surprising finding which leads to numerous applications related to improved health, physiological, intellectual, and emotional states.

### SUMMARY OF THE INVENTION

The present invention encompasses incorporation of oil from transgenic plants engineered to contain significant quantities of stearidonic acid (18:4ω3) for use in human or animal food, feed, supplements, and therapeutics. Sufficient quantities of SDA enriched soybeans have been grown to allow the delivery of soybeans and soy oil with a substantial SDA component. According to embodiments of the current invention, the SDA soybeans of the invention provide enhanced nutritional quality relative to traditional omega-3 alternatives such as flaxseed and lack negative taste and low stability characteristics associated with fish oil. Therefore, a preferred embodiment of this invention comprises an edible product with an increased level of beneficial polyunsaturated fatty acids such as SDA, EPA, and DHA. Surprisingly, significant amounts of DHA were incorporated into tissues through edible compositions supplemented with SDA.

Also according to the current invention, testing of diets comprising stearidonic acid has also been conducted and SDA consumption has resulted in increased DHA concentration of animal tissues, including nervous tissue. A preferred embodiment of the current invention is the usage of the SDA oil produced by transgenic plants in the production of food, feed, supplements, and therapeutics, including applications to both humans and animals. Application of such edible compositions may be particularly preferred in wherein said human or animal lacks ideal DHA concentration in certain tissues.

Embodiments of the invention include methods of improving the physiological condition of a human or animal comprising: identifying a subject in need of DHA supplementation; providing the subject with an edible composition comprising SDA; and, wherein the subject consumes minimum identifiable doses of SDA on at least two occasions and whereby at least one objective indicator of health or function is improved.

Embodiments of the invention also include methods of improving the physiological condition of a human or animal comprising: measuring the DHA concentration in a fluid, tissue, or cell of the human or animal; providing the subject with an edible composition comprising SDA; wherein the subject consumes minimum identifiable does of SDA on at least two occasions and whereby at least one objective indicator of health or function is improved.

Embodiments of the invention also include edible compositions for animal consumption comprising SDA wherein the edible composition is made by a method comprising: providing feed comprising SDA to an animal; harvesting a tissue or fluid comprising lipids from the animal; extracting at least a portion of the lipids from the tissue or fluid; and incorporating at least a portion of the lipids into a food, feed, supplement, or therapeutic for human or animal consumption.

Other features and advantages of this invention will become apparent in the following detailed description of preferred embodiments of this invention, taken with reference to the accompanying tables and figures.

### Definitions

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “edible composition” refers to a composition orally consumable by humans and animals such as food and feed products, supplements, foods for special dietary needs, medical foods, and therapeutics.

As used herein, the term “food” refers to articles used for food or drink for man or other animals, chewing gum, and articles used for components of any such article.

As used herein, the term “animal” refers primarily to livestock animals (such as for example, pigs, cows, sheep, fish and crustaceans) and companion animals (such as for example, dogs, cats, horses).

As used herein, the percent energy intake (“% energy” or “% en”) can be determined by adding up the caloric content of a particular composition and dividing by the total calorie intake of a human or animal. For example, 100 mg daily intake of a specific fatty acid, which has a calorie content of 9 cal/g, for a person consuming a 2000 calorie daily diet would be approximately 0.045% energy derived from that specific fatty acid. In most cases, averages of at least three days would be used for dietary consumption of a specific fatty acid and total dietary intake for such a calculation.

### DETAILED DESCRIPTION OF THE INVENTION

Embodiments of the invention include methods of improving the physiological condition of a human or animal comprising: identifying a subject in need of DHA supplementation; providing the subject with an edible composition comprising SDA; and, wherein the subject consumes minimum identifiable doses of SDA on at least two occasions and whereby at least one objective indicator of health or function is improved.

Embodiments of the invention also include methods of improving the physiological condition of a human or animal comprising: measuring the DHA concentration in a fluid, tissue, or cell of the human or animal; providing the subject with an edible composition comprising SDA; wherein the subject consumes minimum identifiable does of SDA on at least two occasions and whereby at least one objective indicator of health or function is improved.

Alternative embodiments also include methods wherein the edible composition further comprises DHA and wherein the ratio of DHA/SDA is less than about 1.0. Alternative embodiments also include methods wherein the ratio of DHA/SDA is less than about 0.50, less than about 0.10, less than about 0.01 and also include methods wherein the edible composition does not comprise a substantive amount of DHA.

Alternative embodiments also include methods wherein the objective indicator is a neurological condition, including methods wherein the neurological condition is selected from the group of recognized medical conditions consisting of: spinal cord and neurological injury, psychological stress, anxiety disorders, epilepsy, ADHD attention deficit, bipolar disorder, Zellweger syndrome, neurodegenerative diseases, neurological disorder, mood disorder, Alzheimer's disease, dementia, brain damage or impairment, and depression.

Alternative embodiments also include methods wherein the objective indicator of health is a measurable improvement selected from the group consisting of brain development, intelligence, neuroprotection, learning, long term memory, spatial learning, short term memory, behavior, cognition, cognitive function, locomotor activity, psychomotor development, perception, and responsiveness.

Alternative embodiments also include methods wherein the physiological condition is a cardiovascular condition, including methods wherein the cardiovascular condition is selected from the group consisting of cardiac arrhythmia, angina, and myocardial infarction.

Alternative embodiments also include methods wherein the objective indicator of health is a measurable improvement in a risk-associated indicator selected from the group consisting of triglycerides, inflammatory markers, platelet aggregation, adiponectin.

Alternative embodiments also include methods wherein the at least two occasions comprises at least about 30 occasions within a 30 day period.

Alternative embodiments also include methods wherein the SDA consumed provides at least about 0.05%, 0.1%, 0.2%, 0.5%, 1.0%, 2.0% or more of the average daily energy for the human or animal.

Alternative embodiments also include methods wherein the edible composition further comprises GLA. Alternative embodiments also include methods wherein the edible composition has a ratio of SDA/GLA of about 1.0 to about 10.0. Alternative embodiments also include methods wherein the edible composition has a ratio of SDA/GLA of at least about 1.0, 1.3, 1.5, 1.7, 2.0 or more.

Alternative embodiments also include methods wherein the edible composition further comprises DGLA, EPA, and/or DHA.

Alternative embodiments also include methods wherein the edible composition is selected from the group consisting of food, animal feed, food for special dietary needs, supplements, and therapeutics.

Alternative embodiments also include methods wherein the identifying comprises assessing a neurological indicator, including methods wherein the neurological indicator is selected from a group of objectively measurable neurological elements consisting of: intelligence, memory function, and an emotional state.

Alternative embodiments also include methods wherein the identifying comprises measuring the concentration of DHA in a tissue, cell, or fluid of the human or animal.

Alternative embodiments also include methods wherein the identifying comprises measuring the Omega-3 Index of the animal or human.

Alternative embodiments also include methods wherein the identifying comprises utilizing historical information selected from the group consisting of pedigree and parentage. Alternative embodiments also include methods wherein the identifying comprises utilizing genomic data or histological information. Alternative embodiments also include methods wherein the genomic data comprises at least one single-nucleotide polymorphism. Alternative embodiments also include methods wherein the genomic data comprises nucleic acid sequence data.

Alternative embodiments also include methods wherein the consuming SDA on at least two occasions comprises consuming at least once per day for at least 21 days, 30 days, 35 days, 60 days, 90 days, 120 days, 365 days, or even indefinitely.

Alternative embodiments also include methods wherein the consuming comprises multi-modal consuming. Alternative embodiments also include methods wherein the multi-modal consuming comprises consuming SDA in the form of food or feed products and within 24 hours consuming SDA in a concentrated dosage form, including methods wherein the concentrated dosage form is a pill form.

Alternative embodiments also include methods wherein the edible composition further comprises alpha-linolenic acid (ALA), including methods wherein the ALA concentration consumed is less than about 25% of the total fatty acid content of the edible composition. Alternative embodiments also include methods wherein the ratio of concentrations of SDA/ALA consumed is at least about 0.3, 0.5, 0.7, 1.0, 2.0 or more.

Alternative embodiments also include methods wherein the SDA concentration consumed is less than about 35%, less than 25%, less than 15%, less than 5%, and even less than 1% of the total fatty acids in the food or feed.

Alternative embodiments also include methods the edible composition further comprises 6-cis, 9-cis, 12-cis, 15-trans-octadecatetraenoic acid. Alternative embodiments also include methods the edible composition further comprises 9-cis, 12-cis, 15-trans-alpha linolenic acid. Alternative embodiments also include methods the edible composition further comprises 6,9-octadecadienoic acid.

Embodiments of the invention also include an edible composition for animal consumption comprising SDA wherein the edible composition is made by a method comprising: providing feed comprising SDA to an animal; harvesting a tissue or fluid comprising lipids from the animal; extracting at least a portion of the lipids from the tissue or fluid; and incorporating at least a portion of the lipids into a food, feed, supplement, or therapeutic for human or animal consumption.

Alternative embodiments also include edible compositions wherein said feed further comprises gamma linolenic acid (GLA); and additional edible components; and wherein the feed comprises at least about 0.10% stearidonic acid and at least about 0.07% GLA, wherein the ratio of SDA/GLA is at least about 1.0, 1.3, 1.5, 2.0 or more. Alternative embodiments also include edible compositions wherein said SDA concentration in the feed is at least 0.2%, 0.3%, 0.5%, 1.0%, 2%, 3%, 5%, 10%, 15%, 20%, or more. Alternative embodiments also include edible compositions wherein said GLA concentration in the feed is at least about 0.1%, 0.2%, 0.3%, 0.5%, 1%, 2%, 5%, or more.

Alternative embodiments also edible compositions wherein the feed further comprises a transgenic plant product selected from the group consisting of soybeans, soybean oil, soy protein, corn, and canola.

Alternative embodiments also edible compositions wherein the feed further comprises alpha-linolenic acid (ALA). Alternative embodiments also edible compositions wherein the ALA concentration in the feed is less than about 25% of the total fatty acid content of the edible composition. Alternative embodiments also edible compositions wherein the ratio of concentrations of SDA/ALA in the feed is at least about 0.5.

Alternative embodiments also edible compositions wherein the SDA concentration consumed in the feed is less than about 35%, less than 25%, less than 15%, less than 5%, and even less than 1% of the total fatty acids in the feed.

Alternative embodiments also include edible compositions wherein said feed further comprises 6-cis, 9-cis, 12-cis, 15-trans-octadecatetraenoic acid, 9-cis, 12-cis, 15-trans-alpha linolenic acid, and/or 6,9-octadecadienoic acid. Alternative embodiments also include edible compositions wherein said feed further comprises tocochromanol including compositions wherein the tocochromanol is tocopherol.

Alternative embodiments also include edible compositions wherein the lipids comprise an oil.

Alternative embodiments also include edible compositions wherein the animal is a livestock animal. Alternative embodiments also edible compositions wherein the livestock animal is a fish. Alternative embodiments also edible compositions wherein the oil is a fish oil. Alternative embodiments also edible compositions wherein the fish oil has a higher ratio of SDA/DHA concentrations than a fish oil from a similar fish not fed edible compositions comprising SDA.

Alternative embodiments also include edible compositions wherein the food, feed, supplement, or therapeutic for human or animal consumption is an animal feed, including embodiments wherein the animal feed is an aquaculture feed such as a feed for fish. Alternative embodiments also include edible compositions wherein the animal feed is a fish meal.

Alternative embodiments also include edible compositions wherein the food, feed, supplement, or therapeutic for human or animal consumption is a supplement for human consumption, including embodiments wherein the supplement for human consumption comprises fish oil.

Alternative embodiments also include edible compositions wherein the additional feed components are selected from the group consisting of salt, antibiotics, corn, wheat, oats, barley, soybean meal, cottonseed meal, flaxseed meal, sunflower meal, canola meal, wheat middlings, wheat bran, rice bran, corn distiller dried grains, brewers grains, corn gluten meal, corn gluten feed, molasses, rice mill byproduct, corn oil, flax oil, soy protein, palm oil, animal fat, humans or animal fat, restaurant grease, antioxidants, tocochromanols, tocopherols, vitamins, minerals, amino acids, and coccidostats.

### Production of SDA:

The present invention relates to a system for an improved method for the plant based production of stearidonic acid and its incorporation into the diets of humans and animals in an effort to improve health. This production is made possible through the utilization of transgenic plants engineered to produce SDA in sufficiently high yield to so as to allow commercial incorporation into food products. For the purposes of the current invention the acid and salt forms of fatty acids, for instance, butyric acid and butyrate, arachidonic acid and arachidonate, will be considered interchangeable chemical forms.

All higher plants have the ability to synthesize the main 18 carbon PUFA's, linoleic acid (LA) and ALA, and in some cases SDA (C18:4n3, SDA), but few are able to further elongate and desaturate these to produce arachidonic acid (AA), EPA or DHA. Synthesis of EPA and/or DHA in higher plants therefore requires the introduction of several genes encoding all of the biosynthetic enzymes required to convert LA into AA, or ALA into EPA and DHA. Taking into account the importance of PUFAs in human health, the successful production of PUFAs (especially the n-3 class) in transgenic oilseeds, according to the current invention can then provide a sustainable source of these essential fatty acids for dietary use. The “conventional” aerobic pathway which operates in most PUFA-synthesizing eukaryotic organisms, starts with Δ6 desaturation of both LA and ALA to yield γ-linolenic (GLA, 18:3n6) and SDA.

Turning to Table 1, it is important to provide a basis of what constitutes ‘normal’ ranges of oil composition vis-a-vis the oil compositions of the current invention. Table 1 gives examples of fatty acid content of various oils commonly used in food products, expressed as a percentage of total oil.

TABLE 1
STANDARDS FOR FATTY ACID COMPOSITION OF OILS (% OF OIL)

| | Rapeseed | | | | Arachis | | | |
|---|---|---|---|---|---|---|---|---|
| | oil (low | | | | oil | | | |
| Fatty | erucic | Sesame | Soybean | Sunflower | (peanut | Coconut | | |
| acid | acid) | seed oil | oil | seed oil | oil) | oil | Maize oil | Palm oil |
| C6:0 | ND | ND | ND | ND | ND | ND-0.7 | ND | ND |
| C8:0 | ND | ND | ND | ND | ND | 4.6-10.0 | ND | ND |
| C10:0 | ND | ND | ND | ND | ND | 5.0-8.0 | ND | ND |
| C12:0 | ND | ND | ND-0.1 | ND-0.1 | ND-0.1 | 45.1 53.2 | ND-0.3 | ND-0.5 |
| C14:0 | ND-0.2 | ND-0.1 | ND-0.2 | ND-0.2 | ND-0.1 | 16.8-21.0 | ND-0.3 | 0.5-2.0 |
| C16:0 | 2.5-7.0 | 7.9-12.0 | 8.0-13.5 | 5.0-7.6 | 8.0-14.0 | 7.5-10.2 | 8.6-16.5 | 39.3-47.5 |
| C16:1 | ND-0.6 | ND-0.2 | ND-0.2 | ND-0.3 | ND-0.2 | ND | ND-0.5 | ND-0.6 |
| C17:0 | ND-0.3 | ND-0.2 | ND-0.1 | ND-0.2 | ND-0.1 | ND | ND-0.1 | ND-0.2 |
| C17:1 | ND-0.3 | ND-0.1 | ND-0.1 | ND-0.1 | ND-0.1 | ND | ND-0.1 | ND |
| C18:0 | 0.8-3.0 | 4.5-6.7 | 2.0-5.4 | 2.7-6.5 | 1.0-4.5 | 2.0-4.0 | ND-3.3 | 3.5-6.0 |
| C18:1 | 51.0-70.0 | 34.4-45.5 | 17-30 | 14.0-39.4 | 35.0-69 | 5.0-10.0 | 20.0-42.2 | 36.0-44.0 |
| C18:2 | 15.0-30.0 | 36.9-47.9 | 48.0-59.0 | 48.3-74.0 | 12.0-43.0 | 1.0-2.5 | 34.0-65.6 | 9.0-12.0 |
| C18:3 | 5.0-14.0 | 0.2-1.0 | 4.5-11.0 | ND-0.3 | ND-0.3 | ND-0.2 | ND-2.0 | ND-0.5 |
| C20:0 | 0.2-1.2 | 0.3-0.7 | 0.1-0.6 | 0.1-0.5 | 1.0-2.0 | ND-0.2 | 0.3-1.0 | ND-1.0 |
| C20:1 | 0.1-4.3 | ND-0.3 | ND-0.5 | ND-0.3 | 0.7-1.7 | ND-0.2 | 0.2-0.6 | ND-0.4 |
| C20:2 | ND-0.1 | ND | ND-0.1 | ND | ND | ND | ND-0.1 | ND |
| C22:0 | ND-0.6 | ND-1.1 | ND-0.7 | 0.3-1.5 | 1.5-4.5 | ND | ND-0.5 | ND-0.2 |
| C22:1 | ND-2.0 | ND | ND-0.3 | ND-0.3 | ND-0.3 | ND | ND-0.3 | ND |
| C22:2 | ND-0.1 | ND | ND | ND-0.3 | ND | ND | ND | ND |
| C24:0 | ND-0.3 | ND-0.3 | ND-0.5 | ND-0.5 | 0.5-2.5 | ND | ND-0.5 | ND |
| C24:1 | ND-0.4 | ND | ND | ND | ND-0.3 | ND | ND | ND |

Source: CODEX STANDARD FOR NAMED VEGETABLE OILS, CODEX-STAN 210 (Amended 2003, 2005).
ND is non-detectable, defined as ≦0.05%.

More recently, oils from transgenic plants have been created. Some embodiments of the present invention may incorporate products of transgenic plants such as transgenic soybean oil. Transgenic plants and methods for creating such transgenic plants can be found in the literature. See for example, WO2005/021761A1. As shown in Table 2, the composition of the transgenic soy oil is substantially different than that of the accepted standards for soy oil.

TABLE 2
A comparison of transgenic soy oil and traditional soy oil fatty acid
compositions (% of Oil)

| | High | Medium | Low |
|---|---|---|---|
| | SDA | SDA | SDA |
| | Soy Oil | Soy Oil | Soy Oil |
| C14:0 (Myristic) | 0.1 | 0.1 | 0.1 |
| C16:0 (Palmitic)) | 12.5 | 12.3 | 12.1 |
| C16:1 (Palmitoleic) | 0.1 | 0.1 | 0.1 |
| C18:0 (Stearic) | 4.2 | 4.6 | 4.2 |
| C18:1 (Oleic) | 16.0 | 18.7 | 19.4 |
| C18:2 (Linoleic) | 18.5 | 23.9 | 35.3 |
| C18:3 n6 (Gamma Linolenic) | 7.2 | 6.4 | 4.9 |
| C18:3 n3 (Alpha-Linolenic) | 10.3 | 10.8 | 10.1 |
| C18:4 n3 (Stearidonic) | 28.0 | 20.5 | 11.4 |
| C20:0 (Arachidic) | 0.4 | 0.4 | 0.4 |
| C20:1 (Eicosenoic) | 0.3 | 0.2 | 0.4 |
| C22:0 (Behenic) | 0.3 | 0.3 | 0.4 |
| C24:0 (Lignoceric) | 0.1 | 0.1 | 0.1 |
| 6-cis, 9-cis, 12-cis, 15-trans- | <0.2% | <0.2% | <0.2% |
| octadecatetraenoic acid | | | |
| 9-cis, 12-cis, 15-trans-alpha linolenic acid | <0.2% | <0.2% | <0.2% |
| 6,9-octadecadienoic acid | <0.2% | <0.2% | <0.2% |
| Total trans-fatty acid | 1.5 | 1.2 | 0.9 |
| Other fatty acids | 0.6 | 0.6 | 0.3 |

Given the above and according to embodiments of the current invention, the SDA rich soybeans produced in a recombinant oilseed plant provides a composition not previously available for manufacturers. Various embodiments of the invention provide for the incorporation of seed or seed oil into edible compositions for humans or animals with a unique fatty acid profile. In addition, the use of these compositions are made possible without the traditional concerns with stability when oils comprising DHA are delivered from a fish or algal source.

For preferred embodiments of the invention, the preferred source of stearidonic acid is transgenic soybeans which have been engineered to produce high levels of stearidonic acid. The soybeans may be processed at an oil processing facility and oil may be extracted consistent with the methods described in US Patent Applications 2006/0111578A1, 2006/0110521A1, and 2006/0111254A1.

### Methods of Dietary Improvement for Humans or Animals:

Accordingly, in embodiments of the present invention, the methods comprise increasing the levels of Omega-3 fatty acids where SDA is added to said human or animal food in an amount in excess of 0.1% of the food, in excess of 0.2% of the food, in excess of 0.5% of the food, and in excess of 0.8% of the food, where the percentages are based on the total fatty acid concentration of the animal food. In some embodiments, the concentration of SDA may be added to the animal feed in amounts as high as 5% or even 10% of the total fatty acid concentrations. The source of added SDA can be synthetic or natural. The natural SDA is sourced from grain or marine oils or from oils from the group consisting of palm oil, sunflower oil, safflower oil, cottonseed oil, canola oil, corn oil, soybean oil, and flax oil. Preferably, the grain or oilseed is genetically modified to increase the SDA level in such grain or oil as compared to the levels of stearidonic acid found in the native grain or oil.

The SDA may be incorporated in the human or animal diet in the form of a whole seed, ground or cracked seed, extruded seed, extracted oil, triglyceride, or ethyl ester. SDA may be incorporated into the diet and fed to the humans or animals in a meal, oil, crumble, pellet, encapsulated form, whole, cracked, ground or extruded seeds. The SDA may be combined with grains (i.e., corn, wheat, barley), oilseed meals (i.e., soybean meal, cottonseed meal, flaxseed meal, canola meal, sunflower meal), byproducts (i.e., wheat middlings, wheat bran, rice bran, corn distiller dried grains, brewers grains, corn gluten meal, corn gluten feed, molasses, rice mill byproduct), oils (i.e., corn oil, flax oil, soy oil, palm oil, animal fat, fish oil, restaurant grease, and blends thereof), vitamin and minerals, amino acids, antioxidants, tocochromanols, tocopherols, coccidostats and/or antibiotics, enzymes (i.e., phytase, xylanase), emulsifyers, stabilizers, antioxidants or any other food or feed additives.

### Optimum Methods of Incorporation

As shown above, SDA can be used to deliver the benefits of DHA through in vivo processes. However, the effects are expected to be highly dependent on the dosage amount and duration. In particular, a relatively low dosage for a long period of time is likely to be more effective than too small of a dosage or a large dosage in a short period of time.

One measure of dosage that is particularly useful is a percentage of energy intake for a specific fatty acid. Percentage of energy (abbreviated “% en”) can be calculated by dividing the caloric content of the daily consumption of the fatty acid of interest by the total daily caloric consumption.

For example, current estimated intake of EPA and DHA fatty acids in the US derived from the National Health and Nutrition Examination Survey (NHANES 1999-2002) database is about 100 mg per day, which equates to about 0.045% en (1 g of fat has a caloric value of 9). A preferred intake level of EPA and DHA for optimum health is believed to be about 500 mg per day, or about 0.225% en based on a 2000 calorie/day diet. The maximum recommended amount of EPA and DHA is about 3 grams per day or 1.35% en. Few people achieve optimum levels within currently available methods, in part due to the difficulty in obtaining EPA and DHA in typical western diets.

Optimum methods of consumption of Omega 3 fatty acids comprising SDA would include long-term, regular dosages of between 0.045% en and 4.05% en. Dosages of greater than 4.05% en are also likely to incorporate EPA and DHA into the human or animal, but are not likely to produce additional benefit and may in some cases have undesirable side effects.

The preferred range of SDA consumption extends higher than the range for EPA and DHA consumption due to the inherent in vivo conversion efficiency. That is, while some SDA does get converted to EPA and DHA, it is less than the total amount consumed. The estimated upper value of 4.05% en is based on the 1:3 relative enrichment of blood tissues with EPA by EPA vs. SDA. That is, it takes 3 times the amount of SDA as EPA to enrich blood tissues with an equivalent amount of EPA.

The relationship between duration of consumption and deposition of EPA and DHA can clearly be seen by comparing Treatments 5 and 6, in Table 7 above. While not wishing to be bound by theory, the inventors believe that the preferred consumption in terms of percent energy is a function of duration of consumption. Specifically, preferred ranges include consumption of at least about 0.045% en for at least 60 days, 90 days, 120 days, and even 365 days. More preferred ranges include consumption of at least about 0.1% en for at least 30 days, 60 days, 90 days, 120 days, and even 365 days. Additional preferred ranges include consumption of at least about 0.5% en for at least 30 days, 60 days, 90 days, 120 days, and even 365 days. Additional preferred ranges include consumption of at least about 1% en for at least 30 days, 60 days, 90 days, 120 days, and even 365 days. Further preferred ranges include consumption of at least about 2% en for at least about 21 days, 30 days, 60 days, 90 days, 120 days, and even 365 days.

While consistent and regular consumption according to these ranges is believed to be optimum, one may obtain similar benefits even with irregular consumption. In other words, the % energy ranges described herein are not intended to imply the requirement for a precise or fixed daily consumption. Variation in energy intake and normal dietary ranges are likely to yield similar improvements in indicators of health and/or deposition of DHA, provided the average consumption is reasonable within the ranges described herein.

In some cases, such as when a health or neurological condition associated with DHA deposition is identified in a human or animal subject, the subject may even consume edible compositions having increased SDA concentrations indefinitely.

In some cases, optimum consumption of SDA may not be desirable from a single source. In particular, consumption of edible compositions for humans may require variety and other aesthetic considerations which preclude consistent and regular ingestion simply through single-source consumption of SDA. One solution is to incorporate SDA into a concentrated supplement or therapeutic form such as for example, a pill form. Even so, consistent and regular consumption may require large numbers of pills or other concentrated forms to achieve the optimum consumption level. Further alternative methods of consumption include multi-modal ingestion whereby a portion of the SDA is ingested in a concentrated form such as a supplement or therapeutic form and a portion of the SDA is ingested in the form of a supplemented food or feed. For example, incorporating an SDA-containing vegetable oil such as a transgenically modified soybean oil into typical foods could account for a portion of the dietary intake and a further portion could be ingested in the form of a supplement or therapeutic, such as for example, a pill. Given the relatively high dosages required to demonstrate DHA deposition in heart and brain tissues (as described in the examples below) it is believed that multi-mode ingestion may be a key method for delivery of optimum results.

Importantly, such multi-mode ingestion methods may be combined with other sources of omega-3 fatty acids such that EPA and DHA deposition is increased. For example, SDA may supply a portion of the DHA needs of the subject while DHA may be directly ingested for an additional dose. As mentioned above, the taste and smell of DHA sources are traditionally negatives when associated with inclusion in typical western diets. As such, preferred multi-mode and multi-compositional methods would favor SDA inclusion in food and EPA and/or DHA inclusion in supplement or therapeutic form. For such multi-compositional methods, the EPA and DHA could be derived from marine sources, transgenic plant sources, algal sources, or any other source.

Furthermore, in some instances, the need for additional EPA and DHA deposition may be demonstrated through the analysis of genetic or pedigree information, such as for example, in genome-based diet design. An example of genome-based diet design can be found in pct application WO07121396A1.

### Improved Human or Animal Tissues:

Preferred embodiments of the present invention comprise methods of increasing the levels of omega 3 fatty acids, particularly DHA, in the tissues of humans or animals, where the method comprises adding SDA to an edible composition in an amount at least about 0.1%, 0.2%, 0.5%, 0.8% 1.5%, 5%, 10%, 20%, or more, based on the total fatty acid concentration in the food, feed, supplement, or therapeutic. In the case of supplements or therapeutic compositions, the SDA may be even higher including substantially pure forms of SDA, such as for example, ethyl esters of SDA.

### Significance of Increased DHA:

Fatty acids are known to have a significant impact on the health of humans and animals. Numerous studies have examined the impact of specific fatty acids. In some instances, interest in fatty acids has derived from epidemiological studies which have shown correlations between dietary consumption of specific fatty acids and measurable health states. For example, fish and fish oil containing the fatty acids EPA and DHA have been well studied for their ability to improve health states. DHA, in particular, is of interest in numerous neurological studies due to its high concentration in nervous tissue such as the brain.

Examples of conditions frequently associated with DHA consumption include the following: spinal cord and neurological injury, psychological stress, anxiety disorders, epilepsy, ADHD attention deficit, bipolar disorder, Zellweger syndrome, neurodegenerative diseases, Schizophrenia, neurological disorder, mood disorder, Schizochytrium/Schizophrenia, Alzheimer's disease, dementia, brain damage or impairment, and depression.

Furthermore, DHA is involved in many functions related to healthy states of humans and animals. For example, the literature includes investigation of the role of DHA in the following functions: brain development especially in infants and children, intelligence, neuroprotection, learning, spatial learning, memory, behavior, cognition, cognitive function, locomotor activity, psychomotor development, perception, and responsiveness.

Numerous journal articles and patent references related to DHA and associated health benefits are readily available. A sampling of articles on a variety of topics can be found in the references listed below, each of which is herein incorporated by reference.

Subjects in need of DHA supplementation may in many cases simply consume DHA on a short-term basis whereby DHA is then incorporated in to the physiological processes and/or tissues of the body. However, in many cases, long-term consumption of the appropriate fatty acids is required to obtain an optimum result. Consumption of DHA in significant quantities and over long periods of time may be undesirable for taste and odor (such as human consumption of fish oil), or cost reasons (such as, for example, in feeding animals or in low income populations without access to affordable cuts of fish). Dietary supplementation with SDA may alleviate these significant problems while still delivering in vivo DHA to the subject.

Identifying subjects in need of DHA may be based a variety of factors related to the disease states and/or physiological functions mentioned above.

One preferred method of identifying a subject in need of DHA supplementation is called the Omega-3 Index (Harris et al., 2004, herein incorporated by reference). The Omega-3 Index is simply the sum of the EPA, and DHA concentrations in Red Blood Cell (RBC) membrane fatty acids, expressed as a percentage.

### Illustrative Embodiments of the Invention

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

In the examples below, SDA ethyl esters were used in place of traditional oils to isolate the specific fatty acid and allow for higher dosages. Similar results would be obtained when feeding oil derived from transgenic plants such as soy, corn, or canola. Application of ethyl esters of fatty acids is a common practice in the nutritional sciences; see, for example, Krokhan et al., 1993; Arachchige et al., 2006; Martinez et al., 2000; Lim et al., 2000; and Allen et al., 1998, each of which is herein incorporated by reference.

Furthermore, human subjects were not used for this trial for obvious reasons. It is believed that pigs present an adequate model of human physiology with respect to metabolism and deposition of fatty acids. Sampling of human internal organs involves invasive procedures that would require complicated research protocols, ethical review and would likely limit the study population to those diseased individuals already requiring surgery. Use of the pig as an appropriate research model allows for ethical exploration of the effects of SDA on all internal tissues. As is well known in the art, pigs are frequently used as an animal model for human physiology in both the nutritional and medical sciences.

### Edible Compositions:

Various edible composition containing or derived from oils are well known in the art including food products, foods for special dietary needs, supplements, medical foods, and therapeutics. In some cases, these edible compositions contain oils derived from vegetable sources which contain omega-3 fatty acids such as flax oil, black currant oil, borage oil, or echium oil. Other popular oils containing omega-3 fatty acids include fish oils derived from marine sources.

Examples of specific edible compositions derived from transgenic soybean oils comprising SDA can be found in co-pending U.S. provisional applications 60/878300, 60/787301, 60/959910, and US Patent Applications 2006/0111578A1, 2006/0110521A1, and 2006/0111254A1.

Furthermore, edible supplements, foods for special dietary needs, and therapeutics may also be derived which contain oils comprising SDA. Numerous supplement and therapeutic compositions including fatty acids are known in the art. See for example, the following US patents and applications, herein incorporated by reference: US20080020102A1, US20070112071A1, US20070010480A1, US20060228403A1, US20060166935A1, US20060134294A1, US20060105033A1, US20060088574A1, US20050130937A1, US20050101670A1, US20050075399A1, US20050008690A1, US20040076695A1, US20040048926A1, US20040009160A1, US20040001874A1, US20030198730A1, US20030165596A1, US20030077342A1, US20030060509A1, US20030050341A1, US20020198177A1, U.S. Pat. No. 7,001,610, U.S. Pat. No. 6,998,501, U.S. Pat. No. 6,596,302, U.S. Pat. No. 6,576,253, U.S. Pat. No. 6,569,445, U.S. Pat. No. 6,495,599, U.S. Pat. No. 5,866,703, U.S. Pat. No. 5,290,573,

### EXAMPLE 1

### Animal Tissues Incorporating Beneficial Fatty Acids—A 35 Day Study

A 35 day study was conducted to determine whether pigs fed a diet containing SDA could enhance tissues with improved fatty acid profiles including increased levels of SDA, EPA, and DHA.

The dietary treatments are shown in Table3.

TABLE 3
Dietary Treatments for Pigs Fed SDA Enriched Diets.

| Treatment | Description |
|---|---|
| 1 | Control (Corn Oil) (35 days) |
| 2 | 0.2% SDA - Ethyl Esters (35 days) |
| 3 | 0.4% SDA - Ethyl Esters (35 days) |
| 4 | 0.6% SDA - Ethyl Esters (35 days) |
| 5 | 0.8% SDA - Ethyl Esters (35 days) |
| 6 | 0.8% SDA - Ethyl Esters (21 days) |

The percentage levels refer to approximate compositions of SDA in the feed on a gram per gram basis.

A control diet (Treatment 1) formulated to meet or exceed NRC (1998) nutrient requirements for pigs in the late finishing phase (approximately 80-120 kg BW containing 1.14% corn oil and no added SDA ethyl ester (70% w/w SDA) were fed ad libitum during an acclimation period of at least 1 week duration. Ethoxyquin was included in all diets as an antioxidant at a concentration of 150 parts per million (0.015%, as-fed basis), the maximum level allowed by FDA regulation in animal feeds. The same control diet was fed ad libitum to the control pigs (pen receiving Treatment 1) throughout a 35-day test period. Four test diets (Treatment 2, 3, 4, and 5) with SDA ethyl ester substituted for corn oil to provide 0.2, 0.4, 0.6 and 0.8% (w/w SDA active ingredient, as-is basis) were fed ad libitum to one pen of four pigs throughout the 35-day test period Table 4). The ingredient composition of Treatments 1 and 5 are presented in Table 1. All diets were fed in meal form. Treatment 6 was fed the same diet as Treatment 5, but for a duration of only 21 days.

Immediately after diet preparation, samples (approximately 300 g) of each of the five test diets were collected, frozen and stored at approximately 20° C. Samples were tested for dry matter, crude protein and crude fat. On the first and last day of the test period, a sample (˜300 g) of each diet was collected and stored frozen at approximately 20° C. Upon completion of the animal feeding phase of the study, feed samples (mixing, and start and finish of feeding) were analyzed for fatty acids.

All pigs were individually weighed at initiation of the acclimation and test period, and immediately prior to slaughter. Feed consumption per pen was recorded. Upon completion of the feeding period, pigs were slaughtered for tissue collection and carcass evaluation. Pigs were slaughtered using standard industry practices (electrical stunning, exsanguinations, scalding, dehairing and evisceration). Live weights prior to slaughter and hot carcass weights were recorded. The left side of each carcass was fabricated and samples were obtained from the loin (longissimus muscle), ham (semimembranosus muscle) and belly (a section from the center of the belly) for sensory evaluation, fatty acid, and proximate analysis. Prior to removing the sample for sensory and fatty and proximate analysis from the belly the firmness of the belly was evaluated using a flop test. At 24 hr postmortem fat thickness (10th rib), loin eye area (10th rib), visual color, firmness and marbling, Minolta color (L*a*b*), pH, and drip loss were measured.

TABLE 4
Ingredient and calculated composition of diets (as-fed basis)

| Ingredient, % | Diet 1 | Diet 5 |
|---|---|---|
| Corn, Yellow #2 | 88.433 | 88.433 |
| Soybean meal (dehulled) | 7.849 | 7.849 |
| Corn oil | 1.140 | 0 |
| SDA ethyl ester (70% w/w SDA) | 0 | 1.140 |
| Dicalcium phosphate (21:18) | 0.774 | 0.774 |
| Limestone | 0.717 | 0.717 |
| Trace mineral salt^{a} | 0.350 | 0.350 |
| Vitamin mix^{b} | 0.100 | 0.100 |
| L-Lysine | 0.217 | 0.217 |
| DL-Methionine | 0.257 | 0.257 |
| L-Threonine | 0.075 | 0.075 |
| L-Tryptophan | 0.023 | 0.023 |
| Tylan | 0.050 | 0.050 |
| Ethoxyquin | 0.015 | 0.015 |
| Total | 100 | 100□ |
| Calculated nutrients | | |
| ME, kcal/kg | 3395 | 3395 |
| Crude protein, % | 11.72 | 11.72 |
| Crude fat, % | 4.70 | 4.70 |
| Crude fiber, % | 1.93 | 1.93 |
| Ca, % | 0.47 | 0.47 |
| Available P, % | 0.19 | 0.19 |
| Total lysine, % | 0.69 | 0.69 |
| Digestible lysine, % | 0.61 | 0.61 |
| Total Met + Cys, % | 0.69 | 0.69 |
| Digestible Met + Cys, % | 0.64 | 0.64 |
| Digestible threonine, % | 0.41 | 0.41 |
| Digestible tryptophan, % | 0.12 | 0.12 |
| Digestible valine, % | 0.46 | 0.46 |

^{a}Supplemented the following per kilogram of complete diet: Se, 0.30 mg; I, 0.35 mg; Cu, 8 mg; Mn, 20 mg; Fe, 90 mg; Zn, 100 mg; NaCl, 2.8 g.
^{b}Supplemented the following per kilogram of complete diet: vitamin A, 3,307 IU; vitamin D3, 331 IU; vitamin E, 44 mg; vitamin K, 2.2 mg; vitamin B12, 17.9 μg; riboflavin, 4.4 mg; d-pantothenic acid, 12.1 mg, niacin, 16.5 mg; choline chloride, 143 mg.
^{c}Approximation based on assigning the same ME value to SDA ethyl ester as that of corn oil.

Diet 2 was a blend of 75% Diet 1 and 25% Diet 5. Diet 3 was a blend of 50% Diet 1 and 50% Diet 5. Diet 4 was a blend of 25% Diet 1 and 75% Diet 5.
The fatty acid compositions of the diets are presented in Table 5.

TABLE 5
Composition of Test Diets (mg FA per 100 g Feed)

| | Control | | 0.4% | | 0.8% |
|---|---|---|---|---|---|
| | (Corn Oil) | 0.2% SDA | SDA | 0.6% SDA | SDA |
| | Diet 1 | Diet 2 | Diet 3 | Diet 4 | Diet 5 |
| 12:0 | 9.1 | 10.0 | 9.9 | 9.0 | 7.9 |
| 14:0 | 20.3 | 22.3 | 19.8 | 18.6 | 16.5 |
| 16:0 | 601.0 | 592.6 | 541.5 | 477.1 | 429.9 |
| 16:1 n-9 | 0.9 | 1.6 | 0.3 | 0.1 | 0.9 |
| 16:1 n-7 | 5.0 | 5.5 | 4.1 | 3.5 | 3.9 |
| 18:0 | 83.5 | 82.5 | 75.0 | 63.8 | 59.6 |
| 18:1 n-9 | 1035.2 | 1003.3 | 876.3 | 742.0 | 671.7 |
| 18:1 n-7 | 27.1 | 26.8 | 25.2 | 22.2 | 20.1 |
| 18:2 n-6 (LA) | 2393.7 | 2288.8 | 2056.1 | 1767.1 | 1549.5 |
| 18:3 n-6 (GLA) | 1.5 | 21.3 | 41.2 | 58.2 | 66.7 |
| 18:3 n-3 (ALA) | 79.1 | 86.9 | 91.3 | 90.3 | 85.1 |
| 18:4 n-3 (SDA) | 8.1 | 262.1 | 535.7 | 769.3 | 885.6 |
| 20:0 | 16.7 | 23.3 | 31.2 | 36.5 | 39.7 |
| 20:1 n-11 | 0.8 | 1.3 | 2.4 | 0.1 | 0.0 |
| 20:1 n-9 | 10.6 | 9.0 | 6.6 | 0.7 | 0.0 |
| 20:1 n-7 | 0.0 | 0.0 | 0.1 | 0.1 | 0.1 |
| 20:2 n-6 | 0.0 | 0.0 | 0.0 | 0.0 | 0.0 |
| 20:3 n-3 | 0.0 | 0.0 | 0.0 | 0.0 | 0.0 |
| 20:3 n-6 (DGLA) | 0.0 | 0.0 | 0.0 | 0.1 | 0.2 |
| 20:4 n-3 (ETA) | 0.0 | 0.1 | 0.0 | 0.0 | 0.0 |
| 20:4 n-6 (AA) | 0.0 | 0.0 | 2.0 | 3.9 | 5.2 |
| 20:5 n-3 (EPA) | 0.0 | 9.2 | 19.8 | 28.1 | 32.3 |
| 22:0 | 6.7 | 6.2 | 7.0 | 4.9 | 5.2 |
| 22:1 n-11 | 0.0 | 0.0 | 0.0 | 0.0 | 0.0 |
| 22:1 n-9 | 0.0 | 0.0 | 0.0 | 0.0 | 0.0 |
| 22:1 n-13 | 0.0 | 0.0 | 0.0 | 0.6 | 0.4 |
| 22:2 n-7 | 0.0 | 0.0 | 0.0 | 0.0 | 0.0 |
| Std Dev(22:2 n-7) | 0.0 | 0.0 | 0.0 | 0.0 | 0.0 |
| 22:3 n-6 | 0.0 | 0.0 | 0.0 | 1.0 | 0.0 |
| 22:5 n-3 (DPA) | 0.0 | 0.0 | 0.0 | 0.0 | 0.1 |
| 22:6 n-3 (DHA) | 0.0 | 0.0 | 0.0 | 0.0 | 0.0 |
| 24:1 n-9 | 0.0 | 1.9 | 0.0 | 1.6 | 0.0 |
| Total saturates | 737.4 | 737.0 | 684.5 | 610.0 | 558.8 |
| Total monoenes | 1079.5 | 1049.4 | 915.0 | 771.0 | 697.1 |
| Total n-6 | 2395.3 | 2310.2 | 2099.3 | 1830.3 | 1621.6 |
| Total n-3 | 87.2 | 358.3 | 646.9 | 887.7 | 1003.1 |
| Total PUFA | 2482.5 | 2668.5 | 2746.2 | 2718.0 | 2624.7 |
| n-6/n-3 | 27.5 | 6.4 | 3.2 | 2.1 | 1.6 |

In order to evaluate the fatty acid composition of pig tissues (loin, ham, skin, and belly), samples were stored at −80° C. until evaluation. After thawing, ˜22 g of fresh pig tissue was chopped into small pieces and thoroughly mixed before 10 g was sub sampled and lyophilized. Lyophilized samples were ground in 30 ml Sarstedt tubes with two ball bearings using a Mega Grinder. Ground samples (loin and ham: 50 mg; skin: 100 mg; belly: 150 mg; diet: 500 mg) were directly methylated with sulfuric acid in methanol (loin and ham: 1 mL; skin: 3 mL; belly: 10 mL; diet: 1.5 mL) sealed in the presence of butylated hydroxytoluene (50 mg BHT in 100 ml reagent) in reflux conditions of 90° C. Resultant fatty acid methyl esters (FAME's) were separated by capillary gas chromatography (GC) and detected by flame ionization detector (FID). The column used was a Supelco Omegawax 250 capillary column with dimensions of 30 m×0.25 mm×0.25 μm film thickness. The run time was 32 minutes. Peaks were identified based on their relative retention time compared to a FAME reference mixture. Quantification was achieved by using an internal standard (c15:0 triacylglyceride (TAG) for pig tissue samples and c17:0 TAG for diet samples). Results are reported as FA g/100 g fat with theoretical response correction.

For Table 6 below, each value represents g/100 g total fatty acids a mean of 4 samples (1 sample from each of 4 pigs) per treatment.

TABLE 6
Fatty Acid Composition Pork Products - Ham (g FA per 100 g fat)¹

| | Ham (Semi-membranous) | | | | |
|---|---|---|---|---|---|
| Sample Type | 0.2% | | | | |
| Feed | SDA | 0.4% SDA | 0.6% SDA | 0.8% SDA | Control |
| 12:0 | 0.06 | 0.06 | 0.06 | 0.05 | 0.05 |
| 14:0 | 1.06 | 1.10 | 1.05 | 0.96 | 0.92 |
| 16:0 | 21.24 | 21.45 | 21.49 | 20.70 | 20.17 |
| 16:1 n-9 | 0.26 | 0.25 | 0.26 | 0.53 | 0.26 |
| 16:1 n-7 | 3.58 | 3.45 | 3.03 | 2.90 | 3.31 |
| 18:0 | 10.13 | 10.74 | 11.47 | 11.25 | 10.72 |
| 18:1 n-9 | 40.91 | 39.94 | 37.64 | 35.19 | 35.25 |
| 18:1 n-7 | 4.55 | 4.28 | 4.08 | 4.27 | 4.49 |
| 18:2 n-6 (LA) | 8.86 | 9.18 | 9.92 | 11.90 | 13.41 |
| 18:3 n-6 (GLA) | 0.08 | 0.09 | 0.14 | 0.21 | 0.13 |
| 18:3 n-3 (ALA) | 0.20 | 0.26 | 0.22 | 0.25 | 0.21 |
| 18:4 n-3 (SDA) | 0.03 | 0.03 | 0.16 | 0.24 | 0.00 |
| 20:0 | 0.16 | 0.16 | 0.16 | 0.13 | 0.14 |
| 20:1 n-11 | 0.04 | 0.04 | 0.03 | 0.03 | 0.04 |
| 20:1 n-9 | 0.69 | 0.66 | 0.59 | 0.52 | 0.61 |
| 20:1 n-7 | 0.04 | 0.04 | 0.04 | 0.04 | 0.03 |
| 20:2 n-6 | 0.38 | 0.36 | 0.28 | 0.27 | 0.46 |
| 20:3 n-3 | 0.03 | 0.04 | 0.04 | 0.03 | 0.03 |
| 20:3 n-6 (DGLA) | 0.29 | 0.32 | 0.43 | 0.50 | 0.46 |
| 20:4 n-3 (ETA) | 0.09 | 0.14 | 0.26 | 0.29 | 0.01 |
| 20:4 n-6 (AA) | 1.96 | 1.90 | 2.60 | 3.12 | 3.85 |
| 20:5 n-3 (EPA) | 0.17 | 0.23 | 0.54 | 0.87 | 0.07 |
| 22:0 | 0.03 | 0.04 | 0.07 | 0.10 | 0.05 |
| 22:1 n-11 | 0.00 | 0.00 | 0.00 | 0.00 | 0.00 |
| 22:1 n-9 | 0.00 | 0.01 | 0.00 | 0.00 | 0.00 |
| 22:1 n-13 | 0.00 | 0.00 | 0.00 | 0.00 | 0.00 |
| 22:2 n-7 | 0.00 | 0.00 | 0.00 | 0.00 | 0.00 |
| 22:3 n-6 | 0.24 | 0.26 | 0.24 | 0.24 | 0.54 |
| 22:5 n-3 (DPA) | 0.39 | 0.44 | 0.67 | 0.83 | 0.31 |
| 22:6 n-3 (DHA) | 0.09 | 0.08 | 0.10 | 0.13 | 0.05 |
| 24:1 n-9 | 0.04 | 0.05 | 0.06 | 0.07 | 0.07 |
| Total saturates | 32.68 | 33.55 | 34.30 | 33.18 | 32.05 |
| Total monoenes | 50.11 | 48.71 | 45.72 | 43.54 | 44.06 |
| Total n-6 | 11.81 | 12.11 | 13.60 | 16.25 | 18.83 |
| Std Dev(Total n-6) | 0.08 | 0.03 | 0.13 | 0.06 | 0.05 |
| Total n-3 | 1.01 | 1.23 | 1.99 | 2.64 | 0.68 |
| Total PUFA | 12.82 | 13.34 | 15.59 | 18.89 | 19.51 |
| n-6/n-3 | 11.69 | 9.85 | 6.83 | 6.16 | 27.69 |

¹Total fat (g) per 100 g fresh tissue was 3.2, 3.3, 2.2, 1.9, and 2.0 for the 0.2% SDA, 0.4% SDA, 0.6% SDA, 0.8% ASDA and Control, respectively.

Feeding SDA to pigs for the last 35 days prior to slaughter resulted in a significant increase in omega 3 fatty acid enrichment in belly, loin, ham and skin tissues as compared to the control. SDA, ETA, and EPA were enriched in belly, loin, ham, and skin tissues in a dose dependent manner. DPA was enriched in loin and ham tissues in a dose dependent manner. Importantly, DHA was enriched in ham in a dose dependent manner. The SDA supplementation results in higher levels of SDA, ETA, EPA, DPA in belly, loin, ham and skin tissues and DHA in ham tissue as compared to the control.

A comparison of the results for Treatment 5 (35 day) and Treatment six (21 day) suggest a relationship between the duration of the feeding and the incorporation of SDA and DHA. Specifically, 35 days of SDA consumption yielded about 27% more DHA deposition than 21 days of SDA consumption.

TABLE 7
Fatty acid concentrations in semi-membraneous
tissue (e.g., Ham) in mg FA/100 g of fresh tissue

| | Fatty acid | | | | | |
|---|---|---|---|---|---|---|
| | Treatment 1 | Treatment 2 | Treatment 3 | Treatment 4 | Treatment 5 | Treatment 6 |
| | (Control) | (SDA 0.2%) | (SDA 0.4%) | (SDA 0.6%) | (SDA 0.8%) | (SDA 0.8%) |
| | Duration | | | | | |
| | 35 Day | 35 Day | 35 Day | 35 Day | 35 Day | 21 Day |
| Lipid content, % | 2.01 | 3.15 | 3.5 | 2.24 | 1.88 | 3.23 |
| 12:0 | 1.07 | 2.04 | 2.22 | 1.37 | 1.04 | 2.2 |
| 14:0 | 18.8 | 34.0 | 38.2 | 23.9 | 18.3 | 36.8 |
| 16:0 | 401.8 | 673.5 | 748.4 | 482.6 | 386.7 | 701.1 |
| 18:0 | 209.1 | 313.7 | 370.6 | 252.5 | 206.6 | 358.1 |
| Total saturates^{d} | 634.5 | 1029.0 | 1166.5 | 765.2 | 616.6 | 1104.4 |
| 16:1 n-7 | 67.5 | 116.6 | 121.6 | 69.5 | 52.8 | 108.3 |
| 18:1 n-9 | 727.7 | 1303.4 | 1404.9 | 854.9 | 685.9 | 1266.2 |
| 18:1 n-7 | 89.7 | 146.2 | 151.0 | 92.6 | 81.5 | 138.1 |
| 20:1 n-9 | 12.6 | 22.0 | 23.6 | 13.3 | 9.9 | 21.8 |
| 22:1 n-11 | 0.70a | 1.06ab | 1.25b | 0.58a | 0.57a | 0.95ab |
| Total monoenes^{e} | 905 | 1600 | 1715 | 1039 | 846 | 1546 |
| 18:2 n-6 | 236.4 | 264.7 | 316.6 | 215.7 | 208.6 | 290.7 |
| 20:2 n-6 | 8.4ab | 11.4a | 12.7a | 6.1b | 5.1b | 11.3a |
| 20:4 n-6 | 66.4 | 57.4 | 64.3 | 55.3 | 54.2 | 63.4 |
| Total n-6^{f} | 331.0 | 351.2 | 416.4 | 294.5 | 284.6 | 388.1 |
| 18:3 n-3 | 4.12 | 5.60 | 9.17 | 5.03 | 4.70 | 8.66 |
| 18:4 n-3 | 0.00a | 0.76ab | 1.45b | 3.56c | 4.09c | 3.67c |
| 20:3 n-3 | 0.56 | 0.91 | 1.49 | 0.87 | 0.62 | 1.33 |
| 20:4 n-3 | 0.18a | 2.23ab | 5.04bc | 5.85c | 5.36bc | 7.23c |
| 20:5 n-3 | 1.28a | 3.98a | 7.91b | 11.32bc | 14.69c | 9.68b |
| 22:5 n-3 | 5.6a | 11.1b | 15.2c | 14.4bc | 14.2bc | 13.2bc |
| 22:6 n-3 | 1.27 | 2.21 | 2.59 | 2.14 | 2.35 | 1.85 |
| Total n-3 | 13.0a | 26.8a | 42.9b | 43.1b | 46.0b | 45.6b |
| Total PUFA | 344.0 | 377.9 | 459.2 | 337.7 | 330.6 | 433.7 |
| n-3/n-6 | 0.039a | 0.080b | 0.101bc | 0.147d | 0.162d | 0.121c |

a Values reported are least squares means
b^{d}Includes 15:0, 17:0, 20:0, and 22:0
^{e}Includes 16:1n-9, 20:1n-11, 20:1n-7, 22:1n-13, and 24:1
^{f}Includes 18:3n-6, 20:3n-6, and 22:3n-6

### Brain and Heart Fatty Acids:

In addition to measuring long-chain fatty acid incorporation into tissues, additional research was undertaken to understand the alterations in fatty acid concentration in the brain and heart tissues. In particular, DHA is known to be a significant fraction of brain composition. As such, the following experiments were conducted with brain matter extracted from the pigs used in the study above.

After completion of the feeding phase, animals were slaughtered and tissues were collected for evaluation. A sample of brain was obtained by making a cup from the occipital condyle at the base of the skull to the back of the eye. A portion of the brain from the cerebral cortex, and some of the mid brain was collected.

### Phospholipid (PL) FA Composition of Tissues

About 1 g of frozen heart or brain tissue was pulverized in a stainless steel mortar and pestle maintained at −70° C. It was then suspended in 2 mL of ice-cold saline and sonicated on ice to thoroughly disperse the tissue. The lipids were extracted with methylene chloride:methanol. The PL fractions were isolated by thin layer chromatography and methylated for 10 minutes in BF3-methanol at 100° C. After extraction of the fatty acid methyl esters (FAME) with hexane:water, their composition was analyzed in a GC2010 (Shimadzu Scientific Instruments, Columbia, Md.) using a fused silica capillary columns (SP-2560, 100 m length, 0.25 mm internal diameter, 0.25 um film thickness, Supelco, Bellefonte, Pa.). A weighed external standard fatty acid (FA) mixture (GLC727, NuCheck Prep, Elysian, Minn.) was included to correct for potential differences in FA response factors. The response factor for palmitic acid of is assumed to be 1.0. Results were expressed as a percent of total identified FA (for composition endpoints) and as mg FA/g tissue.

### Heart Lipid Deposition:

TABLE 8
Average heart SDA, EPA, and DHA levels after
supplementation with SDA. Fatty acids are reported as
percent of total fatty acids

| | | Treatment | | | |
|---|---|---|---|---|---|
| | Treatment | 2 | Treatment 3 | Treatment 4 | Treatment 5 |
| | 1 | (SDA | (SDA | (SDA | (SDA |
| | Control) | 0.2%) | 0.4%) | 0.6%) | 0.8%) |
| SDA | 0.02% | 0.05% | 0.13% | 0.19% | 0.36% |
| EPA | 0.27% | 1.82% | 3.86% | 4.83% | 6.71% |
| DHA | 0.27% | 0.49% | 0.42% | 0.57% | 0.62% |

SDA feeding resulted in a dose response deposition of SDA, EPA and DHA in heart phospholipids (Table 8). Regression analysis on the mean response at each dose levels demonstrated that the dose response was statistically significant (p<0.05) for deposition of SDA, EPA and DHA in the heart.

### Brain Lipid Deposition:

SDA feeding resulted in a dose response deposition of EPA and DHA in the brain (Table 8).

TABLE 9
Brain phospholipid concentrations of SDA, EPA, and DHA
levels after supplementation with SDA. Fatty acids are
reported as percent of total fatty acids.

| | | Treatment | | | |
|---|---|---|---|---|---|
| | Treatment | 2 | Treatment 3 | Treatment 4 | Treatment 5 |
| | 1 | (SDA | (SDA | (SDA | (SDA |
| | (Control) | 0.2%) | 0.4%) | 0.6%) | 0.8%) |
| SDA | 0.01% | 0.01% | 0.01% | 0.01% | 0.01% |
| EPA | 0.02% | 0.02% | 0.04% | 0.06% | 0.06% |
| DHA | 7.38% | 10.07% | 9.15% | 10.99% | 11.21% |

To further explore the effect of SDA on brain tissue composition, samples from the four control and four high dose (Treatment 5) animals were shaved to isolate the outer cortex. This was done to test the hypothesis that white, myelinated tissue found in the mid brain may not accumulate DHA as readily as gray matter in the cerebral cortex. Results from a total lipid extract of the outer cortex region showed an increase in DHA and EPA after supplementation with SDA (Table 10).

TABLE 10
Brain Phospholipid analysis of the control and 0.8% SDA
(Treatments 1 and 5)

| | Control | 0.8% SDA |
|---|---|---|
| | Average | Average |
| C14:0 | 0.64% | 0.50% |
| C16:0 | 25.33% | 24.20% |
| C16:1n7t | 0.52% | 0.28% |
| C16:1n7 | 1.19% | 1.05% |
| C18:0 | 12.27% | 16.53% |
| C18:1t | 0.13% | 0.08% |
| C18:1n9 | 17.70% | 19.14% |
| C18:2n6t | 0.29% | 0.23% |
| C18:2n6 | 0.83% | 0.63% |
| C18:3n6 | 0.02% | 0.02% |
| C20:1n9 | 0.17% | 0.23% |
| C18:3n3 | 0.11% | 0.15% |
| C18:4n3 | 0.02% | 0.02% |
| C20:2n6 | 0.06% | 0.06% |
| C20:3n6 | 0.28% | 0.35% |
| C20:4n6 | 13.70% | 11.48% |
| C24:0 | 0.04% | 0.02% |
| C20:5n3 | 0.04% | 0.15% |
| C24:1n9 | 0.04% | 0.05% |
| C22:4 | 3.34% | 2.90% |
| C22:5n6 | 6.62% | 3.58% |
| C22:5n3 | 0.39% | 0.98% |
| C22:6n3 | 16.25% | 17.38% |
| OMX-3* | 16.30% | 17.53% |

*Omega 3 Index, i.e. sum of EPA and DHA

### REFERENCES

The references cited in this application, both above and below, are specifically incorporated herein by reference.
1. Allen, P. C. and H. D. Danforth. (1998), Effects of Dietary Supplementation with n-3 Fatty Acid Ethyl Esters on Coccidiosis in Chickens, POULTRY SCIENCE 77:1631-1635.
2. Arachchige Premakumara G.; et al., (2006), Dietary Sesamin and Docosahexaenoic and Eicosapentaenoic Acids Synergistically Increase the Gene Expression of Enzymes Involved in Hepatic Peroxisomal Fatty Acid Oxidation in Rats. METAB. CLIN. EXP., 55:381-90.
3. Bourre J M. (2006) Effects of nutrients (in food) on the structure and function of the nervous system: update on dietary requirements for brain. Part 2: macronutrients. J NUTR HEALTH AGING. 10(5):386-99. Review.
4. Calder, P. C. and Field, C. J. (2002). Fatty Acids, Inflammation and Immunity. IN: NUTRITION AND IMMUNE FUNCTION, (P. C. Calder, C. J. Field and H. S. Gill (Eds)). CABI Publishing. pp: 57-92.
5. Cherian, G. and J. S. Sim. (1995). Dietary α-linilenic acid alters the fatty acid composition of lipid classes in swine tissues. J. AGRICULTURAL FOOD CHEMISTRY 43:2911-16.
6. Enser, M., et al., (2000). Feeding Linseed to Increase the N-3 PUFA of Pork: Fatty Acid Composition of Muscle, Adipose Tissue, Liver and Sausages. MEAT SCIENCE 55:201-12.
7. Fang P C, Kuo H K, Huang C B, Ko T Y, Chen C C, Chung M Y. (2005) The effect of supplementation of docosahexaenoic acid and arachidonic acid on visual acuity and neurodevelopment in larger preterm infants. CHANG GUNG MED J. 28(10):708-15.
8. Freund-Levi Y, et al., (2006) Omega-3 fatty acid treatment in 174 patients with mild to moderate Alzheimer disease: OmegAD study: a randomized double-blind trial. ARCH NEUROL. 63(10): 1402-8.
9. Hashimoto M, Tanabe Y, Fujii Y, Kikuta T, Shibata H, Shido O. (2005) Chronic administration of docosahexaenoic acid ameliorates the impairment of spatial cognition learning ability in amyloid beta-infused rats. J NUTR. 135(3):549-55.
10. Harris W S, et al., (2007) Stearidonic Acid Increases the Red Blood Cell and Heart Eicosapentaenoic Acid Content in Dogs, LIPIDS 42:325-33.
11. Harris W S, et al. (2004) The Omega-3 Index: a new risk factor for death from coronary heart disease? PREVENTIVE MEDICINE 39 212-220.
12. Hirayama S, et al. (2004) Effect of docosahexaenoic acid-containing food administration on symptoms of attention-deficit/hyperactivity disorder—a placebo-controlled double-blind study. EUR J CLIN NUTR. 58(3):467-73.
13. Högyes E, Nyakas C, Kiliaan A, Farkas T, Penke B, Luiten P G. (2003) Neuroprotective effect of developmental docosahexaenoic acid supplement against excitotoxic brain damage in infant rats. NEUROSCIENCE. 119(4):999-1012.
14. Huang W L, King V R, Curran O E, Dyall S C, Ward R E, Lal N, Priestley J V, Michael-Titus A T. (2007) A combination of intravenous and dietary docosahexaenoic acid significantly improves outcome after spinal cord injury. BRAIN. 130(Pt 11):3004-19.(Epub Sep. 27, 2007).
15. James, M. J., Ursin V. M., and Cleland L. G. (2003) Metabolism of stearidonic acid in human subjects: comparison with the metabolism of other n-3 fatty acids. AM J CLIN NUTR 77:1140-5.
16. Judge M P, Harel O, Lammi-Keefe C J. (2007) Maternal consumption of a docosahexaenoic acid-containing functional food during pregnancy: benefit for infant performance on problem-solving but not on recognition memory tasks at age 9 mo. AM J CLIN NUTR. 85(6):1572-7.
17. Kidd P M. (2005) Neurodegeneration from mitochondrial insufficiency: nutrients, stem cells, growth factors, and prospects for brain rebuilding using integrative management. ALTERN MED REV. 10(4):268-93. Review.
18. Klasing, K. C. and Leshchinsky, T. V., (2000), Interactions Between Nutrition and Immunity. Lessons From Animal Agriculture. IN: NUTRITION AND IMMUNOLOGY: PRINCIPLES AND PRACTICE. (M. E. Gershwin, J. B. Germanand, C. L. Keen (Eds)). Elsevier. pp: 363-73.
19. Kotani S, et al.,(2006) Dietary supplementation of arachidonic and docosahexaenoic acids improves cognitive dysfunction. NEUROSCI RES. 56(2):159-64. (Epub Aug. 14, 2006).
20. Kotani S, Sakaguchi E, Warashina S, Matsukawa N, Ishikura Y, Kiso Y, Sakakibara M, Yoshimoto T, Guo J, Yamashima T. (2006) Dietary supplementation of arachidonic and docosahexaenoic acids improves cognitive dysfunction. NEUROSCI RES. 56(2):159-64. (Epub Aug. 14, 2006).
21. Krokhan H E, et al.,(1993). The Enteral Bioavailability of Eicosapentaenoic Acid And Docosahexaenoic Acid is as Good From Ethyl Esters as From Glyceryl Esters in Spite of Lower Hydrolytic Rates by Pancreatic Lipase In Vitro. BIOCHIM BIOPHYS ACTA, 20;1168(1):59-67.
22. Lim S Y, Hoshiba J, Moriguchi T, Salem N Jr. (2005) N-3 fatty acid deficiency induced by a modified artificial rearing method leads to poorer performance in spatial learning tasks. PEDIATR RES. 58(4):741-8.
23. Llorente A M, Jensen C L, Voigt R G, Fraley J K, Berretta M C, Heird W C. (2003) Effect of maternal docosahexaenoic acid supplementation on postpartum depression and information processing. AM J OBSTET GYNECOL. 188(5):1348-53.
24. Logan A C. (2003) Neurobehavioral aspects of omega-3 fatty acids: possible mechanisms and therapeutic value in major depression. ALTERN MED REV. 8(4):410-25. Review.
25. Marangell L B, Martinez J M, Zboyan H A, Kertz B, Kim H F, Puryear L J. (2003) A double-blind, placebo-controlled study of the omega-3 fatty acid docosahexaenoic acid in the treatment of major depression. AM J PSYCHIATRY. 160(5):996-8.
26. Marriott, N. G., et al., (2002). Performance Characteristics and Fatty Acid Composition of Pigs Fed a Diet with Docosahexaenoic Acid. JOURNAL OF MUSCLE FOODS 13:265-77.
27. Martínez M. et al., (2000), Therapeutic effects of docosahexaenoic acid ethyl ester in patients with generalized peroxisomal disorders. AMERICAN JOURNAL OF CLINICAL NUTRITION, Vol.71, No. 1, 376S-385S.
28. Martinez M. (2001) Restoring the DHA levels in the brains of Zellweger patients. J MOL NEUROSCI. 16(2-3):309-16; discussion 317-21. Review.
29. Matthews, K. R., et al., (2000). Effect of whole linseed (Linum usitatissimum) in the diet of finishing pigs on growth performance and on the quality and fatty acid composition of various tissues. BRITISH JOURNAL OF NUTRITION 83:637-643.
30. Miles E A, Banerjee T. and Calder, P. C. (2004), The Influence of Different Combinations of Gamma-Linolenic Acid, Stearidonic Acid and EPA on Immune Function in Healthy Young Male Subjects. BR J NUTR. 91(6):893-903.
31. Michael-Titus A T. (2007) A combination of intravenous and dietary docosahexaenoic acid significantly improves outcome after spinal cord injury. BRAIN. 130(Pt 11):3004-19. (Epub Sep. 27, 2007).
32. Morgan, C., et al., (1992). Manipulation of the fatty acid composition of pig meat lipids by dietary means. J. SCI. FOOD AGRIC. 58:357-368.
33. Morris, S., et al. (1999) Developmental Sensitivity of the Piglet Brain to Docosahexanoic Acid. PEDIATRIC RESEARCH Vol. 46, No. 4, 401-405.
34. Napier, Johnathan. (2007) The Production of Unusual Fatty Acids in Transgenic Plants. ANNU. REV. PLANT BIOL., 58:295-319.
35. Nurnberg, K. U., et al., (1999). Influence of Exogenous Application of n-3 Fatty Acids on Meat Quality, Lipid Composition, and Oxidative Stability in Pigs. ARCH. ANIM. NUTR. 52:53-65.
36. Otten, W., et al., (1993). A High Omega 3 Fatty Acid Diet Alters Fatty Acid Composition of Heart, Liver, Kidney, Adipose Tissue and Skeletal Muscle in Swine. ANN. NUTR. METAB. 37:134-41.
37. Sakakibara M, Yoshimoto T, Guo J, Yamashima T. (2006) Dietary supplementation of arachidonic and docosahexaenoic acids improves cognitive dysfunction. NEUROSCI RES. 56(2):159-64. (Epub Aug. 14, 2006).
38. Schaefer et al., (2006) Plasma phosphatidylcholine docosahexaenoic acid content and risk of dementia and Alzheimer disease: the Framingham Heart Study. ARCH NEUROL. 63(11):1545-50.
39. Specht-Overholt, S., et al., (1997). Fatty Acid Composition of Commercially Manufactured Omega-3 Enriched Pork Products, Haddock, and Mackerel. J. ANIM. SCI. 75:2335-2343.
40. Sun-Young Lim and Hiramitsu Suzuki. (2000), Intakes of Dietary Docosahexaenoic Acid Ethyl Ester and Egg Phosphatidylcholine Improve Maze-Learning Ability in Young and Old Mice. JOURNAL OF NUTRITION. 130:1629-32.
41. Tanaka K, Shimizu T, Ohtsuka Y, Yamashiro Y, Oshida K. (2007) Early dietary treatments with Lorenzo's oil and docosahexaenoic acid for neurological development in a case with Zellweger syndrome. BRAIN DEV. 29(9):586-9. (Epub Apr. 5, 2007).
42. Ursin G. et al., (2003), Modification of Plant Lipids for Human Health: Development of Functional Land-Based Omega-3 Fatty Acids. J. NUTR. 133:4271-74.
43. Vancassel S, Blondeau C, Lallemand S, Cador M, Linard A, Lavialle M, Dellu-Hagedorn F. (2007) Hyperactivity in the rat is associated with spontaneous low level of n-3 polyunsaturated fatty acids in the frontal cortex. BEHAV BRAIN RES. 180(2):119-26. (Epub Feb. 25, 2007).
44. Voigt R G, Llorente A M, Jensen C L, Fraley J K, Berretta M C, Heird W C. (2001) A randomized, double-blind, placebo-controlled trial of docosahexaenoic acid supplementation in children with attention-deficit/hyperactivity disorder. J PEDIATR. 139(2):189-96.
45. Xiao Y, Wang L, Xu R J, Chen Z Y. (2006) DHA depletion in rat brain is associated with impairment on spatial learning and memory. BIOMED ENVIRON SCI. 19(6):474-80.
46. Yuen A W, Sander J W, Fluegel D, Patsalos P N, Bell G S, Johnson T, Koepp M J. (2005) Omega-3 fatty acid supplementation in patients with chronic epilepsy: a randomized trial. EPILEPSY BEHAV. 7(2):253-8.

What is claimed is:

1. A method of improving the physiological condition of a human or animal comprising:
a. identifying a subject in need of DHA supplementation;
b. providing said subject with an edible composition comprising SDA; and,
wherein said subject consumes minimum identifiable doses of SDA on at least two occasions and whereby at least one objective indicator of health or function is improved.

2. The method of claim 1 wherein said edible composition further comprises DHA and wherein the ratio of DHA/SDA is less than about 1.0.

3. The method of claim 1 wherein said objective indicator is a neurological condition.

4. The method of claim 1 wherein said physiological condition is a cardiovascular condition.

5. The method of claim 1 wherein said at least two occasions comprises at least about 30 occasions within a 30 day period.

6. The method of claim 1 wherein said edible composition further comprises GLA.

7. The method of claim 1 wherein said edible composition further comprises DGLA.

8. The method of claim 1 wherein said edible composition further comprises EPA.

9. The method of claim 1 wherein said identifying comprises assessing a neurological indicator.

10. The method of claim 1 wherein said identifying comprises measuring the Omega-3 Index of said animal or human.

11. The method of claim 1 wherein said consuming SDA on at least two occasions comprises consuming at least once per day for at least 21 days.

12. The method of claim 1 wherein said consuming comprises multi-modal consuming.

13. The method of claim 1 wherein said edible composition further comprises alpha-linolenic acid (ALA).

14. The method of claim 1 wherein the ratio of concentrations of SDA/ALA consumed is at least about 0.5.

15. The method of claim 1 wherein the SDA concentration consumed is less than about 35% of the total fatty acids in the food or feed.

16. The method of claim 1 wherein said edible composition further comprises an acid, wherein the acid is selected from the group consisting of 6-cis, 9-cis, 12-cis, 15-trans-octadecatetraenoic acid; 9-cis, 12-cis, 15-trans-alpha linolenic acid; and 6,9-octadecadienoic acid.

17. A method of improving the physiological condition of a human or animal comprising:
a. measuring the DHA concentration in a fluid, tissue, or cell of said human or animal;
b. providing said subject with an edible composition comprising SDA;
wherein said subject consumes minimum identifiable does of SDA on at least two occasions and whereby at least one objective indicator of health or function is improved.

18. The method of claim 17 wherein said edible composition further comprises DHA and wherein the ratio of DHA/SDA is less than about 1.0.

19. The method of claim 17 wherein said objective indicator of health comprises an indicator of a neurological condition.

20. The method of claim 17 wherein said physiological condition is a cardiovascular condition.

21. The method of claim 17 wherein said at least two occasions comprises at least about 30 occasions within a 30 day period.

22. The method of claim 17 wherein said edible composition further comprises GLA.

23. The method of claim 17 wherein said edible composition further comprises DGLA.

24. The method of claim 17 wherein said edible composition further comprises EPA.

25. The method of claim 17 wherein said measuring comprises assessing a neurological indicator.

26. The method of claim 17 wherein said measuring comprises measuring the Omega-3 Index of said animal or human.

27. The method of claim 17 wherein said consuming SDA on at least two occasions comprises consuming at least once per day for at least 21 days.

28. The method of claim 17 wherein said consuming comprises multi-modal consuming.

29. The method of claim 17 wherein said edible composition further comprises alpha-linolenic acid (ALA).

30. The method of claim 17 wherein the ratio of concentrations of SDA/ALA consumed is at least about 0.5.

31. The method of claim 17 wherein the said SDA concentration consumed is less than about 35% of the total fatty acids in the food or feed.

32. The method of claim 17 wherein said edible composition further comprises an acid, wherein the acid is selected from the group consisting of 6-cis, 9-cis, 12-cis, 15-trans-octadecatetraenoic acid; 9-cis, 12-cis, 15-trans-alpha linolenic acid; and 6,9-octadecadienoic acid.

33. An edible composition for animal consumption comprising SDA wherein said edible composition is made by a method comprising:
a. providing feed comprising SDA to an animal;
b. harvesting a tissue or fluid comprising lipids from said animal;
c. extracting at least a portion of said lipids from said tissue or fluid; and
d. incorporating at least a portion of said lipids into a food, feed, supplement, or therapeutic for human or animal consumption.

34. The edible composition of claim 33 wherein said feed further comprises:
a. gamma linolenic acid (GLA);
b. additional edible components; and
wherein said feed comprises at least about 0.10% stearidonic acid and at least about 0.07% GLA, wherein the ratio of SDA/GLA is at least about 1.3.

35. The edible composition of claim 33 wherein said feed further comprises alpha-linolenic acid (ALA).

36. The edible composition of claim 33 wherein the said SDA concentration consumed in said feed is less than about 35% of the total fatty acids in the feed.

37. The edible composition of claim 33 wherein said feed further comprises an acid, wherein the acid is selected from the group consisting of 6-cis, 9-cis, 12-cis, 15-trans-octadecatetraenoic acid; 9-cis, 12-cis, 15-trans-alpha linolenic acid; and 6,9-octadecadienoic acid.

38. The edible composition of claim 33 wherein said feed further comprises tocochromanol.

39. The method of claim 33 wherein said lipids comprise an oil.

40. The method of claim 33 wherein said animal is a livestock animal.
